(19) United States
(12) Patent Application Publication
(10) Pub. No.: US 2025/0151972 A1
(43) Pub. Date: May 15, 2025

(54) Shoe-Cleaning Device

(71) Applicant: Barbara Taylor, South Charleston, WV (US)

(72) Inventor: Barbara Taylor, South Charleston, WV (US)

(21) Appl. No.: 18/655,679

(22) Filed: May 6, 2024

Related U.S. Application Data
(60) Provisional application No. 63/597,493, filed on Nov. 9, 2023.

(51) Int. Cl. A47L 23/18; A61L 9/20; A61L 9/22

(52) U.S. Cl. CPC A47L 23/18; A61L 9/20; A61L 9/22; A61L 2209/12

(57) ABSTRACT

A shoe-cleaning device is provided. The device is comprised of a shoe-cleaning device that can enclose a shoe and eliminate bacteria and odors from said shoe in a self-container manner. To use the device, at least one pair of shoes can be placed into the device and a lid of the device can be closed around the shoes to seal the shoes within the body. Then, an air purifier, a UV light, and at least one fan can be activated via at least one button to disinfect (i.e., kill bacteria) and freshen (i.e., apply scent to) the shoes.

### CROSS-REFERENCE TO RELATED APPLICATION

The present application claims priority to, and the benefit of, U.S. Provisional Application No. 63/597,493, which was filed on Nov. 9, 2023, and is incorporated herein by reference in its entirety.

### FIELD OF THE INVENTION

The present invention relates generally to the field of shoe-cleaning devices. More specifically, the present invention relates to a shoe-cleaning device that can enclose a shoe and eliminate bacteria and odors from said shoe in a self-contained manner. Accordingly, the present disclosure makes specific reference thereto. Nonetheless, it is to be appreciated that aspects of the present invention are also equally applicable to other like applications, devices, and methods of manufacture.

### BACKGROUND

After spending time outdoors, shoes will accumulate dirt, becoming unclean and unsanitary. Furthermore, shoes absorb sweat, especially in hot weather or during sports activities. The heat and moisture create a conducive environment for the growth of bacteria and fungi, which can cause unpleasant odors that often emanate from used footwear. The dampness within the shoes can also compromise the integrity of the material, leading to quicker wear and tear.

When said shoes are then taken off and brought into the home to dry, they can release an unpleasant smell. The smell can linger in the air, causing a nuisance and potentially affecting the indoor air quality. This issue is particularly pronounced in smaller living spaces or in homes with limited ventilation, where the odors are more concentrated and less likely to dissipate quickly.

Additionally, many people find themselves facing a dilemma when their shoes reach this state of uncleanliness and odor. The prospect of cleaning the shoes can be daunting, especially when the dirt and smell are deeply ingrained. The time and effort required to restore the shoes to a wearable state can be discouraging, leading some to consider the option of discarding the shoes altogether.

In such instances, a user may decide to throw the shoes away, believing that no other viable alternative exists. The inconvenience of dealing with dirty, smelly shoes, coupled with the perceived difficulty of cleaning them, can make disposal seem like the easiest solution. This, however, contributes to increased waste and environmental issues, as well as additional costs for the individual in replacing the discarded footwear.

Therefore, there exists a long-felt need in the art for a shoe-cleaning device. There also exists a shoe-cleaning device that allows a user to clean dirty shoes. More specifically, there exists a long-felt need in the art for a shoe-cleaning device that eliminates odors and bacteria from used shoes. Further, there exists a long-felt need in the art for a shoe-cleaning device that eliminates odors and bacteria from used shoes wherein the device does not allow the odor from said shoes to permeate nearby areas.

The subject matter disclosed and claimed herein, in one embodiment thereof, comprises a shoe-cleaning device. The device is comprised of a shoe-cleaning device that can enclose a shoe and eliminate bacteria and odors from said shoe in a self-contained manner. To use the device, at least one pair of shoes can be placed into the device and a lid of the device can be closed around the shoes to seal the shoes within the body. Then, an air purifier, a UV light, and at least one fan can be activated via at least one button to disinfect (i.e., kill bacteria) and freshen (i.e., apply scent to) the shoes.

In this manner, the shoe-cleaning device of the present invention accomplishes all the forgoing objectives and provides a shoe-cleaning device. More specifically, the device allows a user to clean dirty shoes by eliminating odors and bacteria from used shoes. Further, the device does so by not allowing said odor from said shoes to permeate nearby areas.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a shoe-cleaning device. The body of the device can receive at least one pair of shoes, wherein at least one lid can enclose the shoes within the body while the device disinfects and freshens the shoes.

The body is preferably comprised of at least one receiving area that receives at least one pair of shoes. In the event that the shoes are wet, muddy, etc., the receiving area may be comprised of at least one drainage hole in fluid connection with at least one container within the body. The container collects runoff liquid/debris from the shoes that fall through the drainage hole, wherein the container can then be removed from the body and the contents of the container can be disposed of. The body is also preferably comprised of at least one lid that fully encloses the shoes (which have been placed on the receiving area) within the body.

The device is also comprised of at least one air purifier that eliminates odors and bacteria from the shoes once the shoes have been placed on the receiving area and sealed within the body by closing the lid. Bacteria on the shoes may also be killed via at least one UV light that may be positioned on the underside of the lid. The device may also dry the shoes via at least one fan, wherein air can enter/exit the fan via at least one vent hole of the body and/or lid. To further freshen the shoes, the lid or other portion of the body may have at least one scent dispenser that distributes scent within the body and onto the shoes. The scent distributed may be any smell and scent type known in the art.

The present invention is also comprised of a method of using the device. First, a device is provided comprised of a body comprised of at least one receiving area and at least one lid. Then, at least one pair of shoes can be placed on the receiving area. Next, the lid can be closed around the shoes to seal the shoes within the body. Then, an air purifier, a UV light, and at least one fan can be activated via at least one button to disinfect (i.e., kill bacteria) and freshen (i.e., apply scent to) the shoes. Finally, a user can open the lid and remove the shoes once disinfected and freshened.

Accordingly, the shoe-cleaning device of the present invention is particularly advantageous as it provides a shoe-cleaning device. More specifically, the device allows a user to clean dirty shoes by eliminating odors and bacteria from used shoes. Further, the device does so by not allowing said odor from said shoes to permeate nearby areas. In this manner, the shoe-cleaning device provides a solution to easily eliminate bacteria and odors from shoes.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a shoe-cleaning device of the present invention in accordance with the disclosed architecture; and

FIG. 2 illustrates a flowchart of a method of using one potential embodiment of a shoe-cleaning device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a shoe-cleaning device. There also exists a shoe-cleaning device that allows a user to clean dirty shoes. More specifically, there exists a long-felt need in the art for a shoe-cleaning device that eliminates odors and bacteria from used shoes. Further, there exists a long-felt need in the art for a shoe-cleaning device that eliminates odors and bacteria from used shoes wherein the device does not allow the odor from said shoes to permeate nearby areas.

The present invention, in one exemplary embodiment, is comprised of a shoe-cleaning device. The body of the device can receive at least one pair of shoes, wherein at least one lid can enclose the shoes within the body while the device disinfects and freshens the shoes.

The body is preferably comprised of at least one receiving area that receives at least one pair of shoes. In the event that the shoes are wet, muddy, etc., the receiving area may be comprised of at least one drainage hole. The hole is in fluid connection with at least one container within the body that collects runoff liquid/debris from the shoes that fall through the drainage hole. Then, the container can then be removed from the body and the contents of the container can be disposed of. The body is also preferably comprised of at least one lid that fully encloses the shoes (which have been placed on the receiving area) within the body.

The device is also comprised of at least one air purifier that eliminates odors and bacteria from the shoes once the shoes have been placed on the receiving area and sealed within the body by closing the lid. Bacteria on the shoes may also be killed via at least one UV light that may be positioned on the underside of the lid. The device may also dry the shoes via at least one fan, wherein air can enter/exit the fan via at least one vent hole of the body and/or lid. To further freshen the shoes, the lid or other portion of the body may have at least one scent dispenser that distributes scent within the body and onto the shoes. The scent distributed may be any smell and scent type known in the art.

The present invention is also comprised of a method of using the device. First, a device is provided comprised of a body comprised of at least one receiving area and at least one lid. Then, at least one pair of shoes can be placed on the receiving area. Next, the lid can be closed around the shoes to seal the shoes within the body. Then, an air purifier, a UV light, and at least one fan can be activated via at least one button to disinfect (i.e., kill bacteria) and freshen (i.e., apply scent to) the shoes. Finally, a user can open the lid and remove the shoes once disinfected and freshened.

Accordingly, the shoe-cleaning device of the present invention is particularly advantageous as it provides a shoe-cleaning device. More specifically, the device allows a user to clean dirty shoes by eliminating odors and bacteria from used shoes. Further, the device does so by not allowing said odor from said shoes to permeate nearby areas. In this manner, the shoe-cleaning device provides a solution to easily eliminate bacteria and odors from shoes.

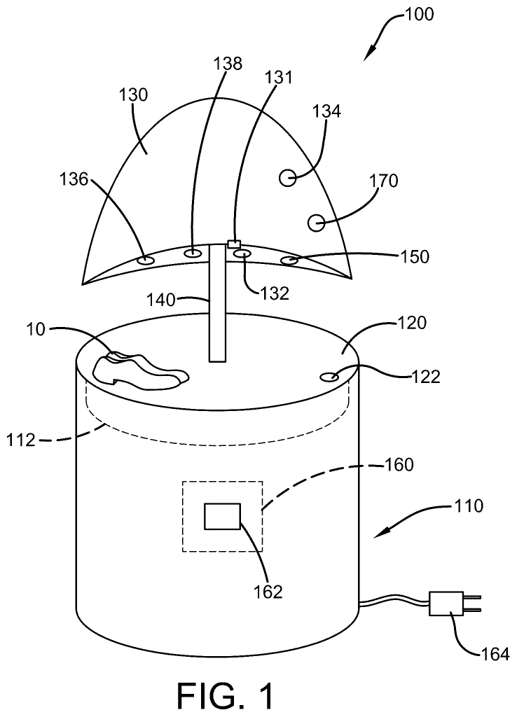

Referring initially to the drawings, FIG. 1 illustrates a perspective view of one potential embodiment of a shoe-cleaning device 100 of the present invention in accordance with the disclosed architecture. The body 110 of the device 100 can receive at least one pair of shoes 10. More specifically, at least one lid 130 can enclose the shoes 10 within the body 110 such that the device 100 can disinfect and freshen the shoes 10.

The body 110 may be any shape, size, and configuration in different embodiments. The body 110 is preferably comprised of at least one receiving area 120 that may be located anywhere on the body 110. The receiving area 120 is preferably flat such that at least one pair of shoes 10 can be placed flat atop the receiving area 120. In the event that the shoes 10 are wet, muddy, etc., the receiving area 120 may be comprised of at least one drainage hole 122. The hole 122 is in fluid connection with at least one container 112 within the body 110 positioned underneath the receiving area 120. The container 112 collects runoff liquid/debris from the shoes 10 that falls through the drainage hole 122, wherein the container 112 can then be removed from the body 110 and the contents of the container can be disposed of.

The body 110 is also preferably comprised of at least one lid 130. The lid 130 may be any shape that allows the lid 130 to fully enclose the shoes 10 (which have been placed on the receiving area 120) within the body 110. The lid 130 can preferably be raised and lowered over the receiving area 120 via at least one support member 140 that the lid 130 can travel up and down upon. The member 140 may be collapsible in height in one embodiment. The lid 130 may be secured to the body 110 via at least one locking member 131. The locking member 131 may be any lock type or locking mechanism known in the art. In one embodiment, the support member 140 is any hinge-type known in the art.

The device 100 is also comprised of at least one air purifier 132. The purifier 132 is preferably an ionic air purifier. The purifier 132 eliminates odors and bacteria from the shoes 10 once the shoes 10 have been placed on the receiving area 120 and sealed within the body 110 via closing the lid 130. Bacteria on the shoes 10 may also be killed via at least one UV light 138 that may be positioned on the underside of the lid 130.

The device 100 may also dry the shoes 10 via at least one fan 136, wherein air can enter/exit the fan 136 via at least one vent hole 134 of the body 110 and/or lid 130. To further freshen the shoes 10, the lid 130 or other portion of the body 110 may have at least one scent dispenser 150. The dispenser 150 may be comprised of a scented sponge, a scented air diffuser, etc. that distributes scent within the body 110 and onto the shoes 10. The scent distributed may be any smell and scent type known in the art.

The functions of the purifier 132, fan 136, and light 138 may be controlled via at least one button 170 located on the body 110 and/or lid 130. The purifier 132, fan 136, and light 138 may also be powered by at least one battery 160. In one embodiment, the battery 160 may be a replaceable battery. In another embodiment, the battery 160 is a rechargeable battery. In one embodiment, the battery 160 can be recharged via at least one charging port 162, such as but not limited to a USB port. The battery 160 may also be charged via at least one male 110v/220v power cord.

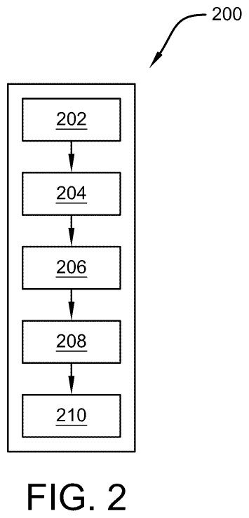

The present invention is also comprised of a method of using 200 the device 100, as seen in FIG. 2. First, a device 100 is provided comprised of a body 110 comprised of at least one receiving area 120, at least one lid 130, an air purifier 132, a UV light 138, and at least one fan 136 can be all be activated via at least one button 170 [Step 202]. Then, at least one pair of shoes 10 (or any number of shoes 10) can be placed on the receiving area 120 [Step 204]. Next, the lid 130 can be closed around the shoes 10 to seal the shoes 10 within the body 110 [Step 206]. Then, an air purifier 132, a UV light 138, and at least one fan 136 can be activated via at least one button 170 to disinfect (i.e., kill bacteria) and freshen (i.e., apply scent to) the shoes 10 [Step 208]. Finally, a user can open the lid 130 and remove the shoes 10 once disinfected and freshened [Step 210].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “shoe-cleaning device” and “device” are interchangeable and refer to the shoe-cleaning device 100 of the present invention.

Notwithstanding the forgoing, the shoe-cleaning device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the shoe-cleaning device 100 as shown in the FIGS. are for illustrative purposes only, and that many other sizes and shapes of the shoe-cleaning device 100 are well within the scope of the present disclosure. Although the dimensions of the shoe-cleaning device 100 are important design parameters for user convenience, the shoe-cleaning device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A shoe-cleaning device comprising:
a body comprised of a receiving area;
a lid;
an air purifier;
a fan; and
a UV light.

2. The shoe-cleaning device of claim 1, wherein the lid attaches to the receiving area via a support member.

3. The shoe-cleaning device of claim 2, wherein the lid is repositionable along the support member.

4. The shoe-cleaning device of claim 1, wherein the lid is comprised of a locking member.

5. The shoe-cleaning device of claim 2, wherein the support member is comprised of a hinge.

6. The shoe-cleaning device of claim 1, wherein the air purifier is comprised of an ionic air purifier.

7. The shoe-cleaning device of claim 1, wherein the lid is comprised of a vent hole.

8. A shoe-cleaning device comprising:
a body comprised of a receiving area comprised of a drainage hole, and a container positioned below the receiving area;
a lid;
an air purifier;
a scent dispenser;
a fan; and
a UV light.

9. The shoe-cleaning device of claim 8, wherein the container is removable from the body.

10. The shoe-cleaning device of claim 8, wherein the drainage hole is in fluid communication with the container.

11. The shoe-cleaning device of claim 8, wherein the air purifier is comprised of an ionic air purifier.

12. The shoe-cleaning device of claim 8, wherein the lid is comprised of a vent hole.

13. The shoe-cleaning device of claim 8, wherein the scent dispenser is comprised of a scented sponge.

14. The shoe-cleaning device of claim 8, wherein the scent dispenser is comprised of a scented air diffuser.

15. The shoe-cleaning device of claim 8, wherein the UV light is positioned on an underside of the lid.

16. The shoe-cleaning device of claim 8 further comprised of a battery.

17. The shoe-cleaning device of claim 16, wherein the battery is comprised of a charging port.

18. A method of using a shoe-cleaning device, the method comprising the following steps:
providing a shoe-cleaning device comprised of a body comprised of a receiving area, a lid, an air purifier, a UV light, a fan, and a button;
placing a shoe on the receiving area;
closing the lid around the shoe;
activating the air purifier, the UV light, and the fan via the button; and
opening the lid and removing the shoe.

19. The method of using a shoe-cleaning device of claim 18, wherein the shoe-cleaning device is comprised of a scent dispenser.

20. The shoe-cleaning device of claim 19, wherein the scent dispenser is comprised of a scented sponge or a scented air diffuser.
